(19) United States
(12) Patent Application Publication
(10) Pub. No.: US 2010/0041646 A1
(43) Pub. Date: Feb. 18, 2010

(54) Phenylene-Bis-Oxazolidine Derivatives and Their Use as Anticoagulants

(75) Inventors: Susanne Rohrig, Hilden (DE); Jens Pohlmann, Basel (CH); Sabine Arndt, Dortmund (DE); Mario Jeske, Solingen (DE); Metin Akbaba, Ratingen (DE); Elisabeth Perzborn, Wuppertal (DE); Christoph Gerdes, Leverkusen (DE); Karl-Heinz Schlemmer, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: BAYER HEALTHCARE AG, Leverkusen (DE)

(21) Appl. No.: 12/084,267

(22) PCT Filed: Oct. 20, 2006
(86) PCT No.: PCT/EP2006/010129

§ 371 Date: Sep. 14, 2009

(30) Foreign Application Priority Data
Nov. 2, 2005 (DE) 102005052174.6

(51) Int. Cl. A61K 31/422; C07D 413/14; A61K 31/5355; A61P 7/02; C12N 5/00

(52) U.S. Cl. 514/228.8; 548/231; 544/96; 514/376; 435/375

(57) ABSTRACT

The present application relates to novel 1,4-phenylene-bis-oxazolidine derivatives, to processes for their preparation, to their use for the treatment and/or prophylaxis of diseases and also to their use for preparing medicaments for the treatment and/or prophylaxis of diseases, in particular thromboembolic disorders.

The present application relates to novel 1,4-phenylene-bis-oxazolidine derivatives, to processes for their preparation, to their use for the treatment and/or prophylaxis of diseases and also to their use for preparing medicaments for the treatment and/or prophylaxis of diseases, in particular thromboembolic disorders.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a joint reaction path, are distinguished. Here factor Xa, which is formed from the proenzyme factor X, plays a key role, since it connects the two coagulation paths. The activated serine protease Xa cleaves prothrombin to thrombin. The resulting thrombin, in turn, cleaves fibrinogen to fibrin. Subsequent crosslinking of the fibrin monomers causes formation of blood clots and thus haemostasis. In addition, thrombin is a potent effector of platelet aggregation which likewise contributes significantly to haemostasis.

Haemostasis is subject to a complex regulatory mechanism. Uncontrolled activation of the coagulation system or defective inhibition of the activation processes may cause formation of local thrombi or embolisms in vessels (arteries, veins, lymph vessels) or in heart cavities. This may lead to serious thromboembolic disorders. In addition, in the case of consumption coagulopathy, hypercoagulability may—systemically—result in disseminated intravascular coagulation. Thromboembolic complications furthermore occur in microangiopathic haemolytic anaemias, extracorporeal blood circulation, such as haemodialysis, and also in connection with prosthetic heart valves.

Thromboembolic disorders are the most frequent cause of morbidity and mortality in most industrialized countries [Heart Disease: A Textbook of Cardiovascular Medicine, Eugene Braunwald, 5th edition, 1997, W.B. Saunders Company, Philadelphia].

The anticoagulants, i.e. substances for inhibiting or preventing blood coagulation, which are known from the prior art, have various, often grave disadvantages. Accordingly, in practice, an efficient treatment method or prophylaxis of thromboembolic disorders is very difficult and unsatisfactory.

In the therapy and prophylaxis of thromboembolic disorders, use is firstly made of heparin, which is administered parenterally or subcutaneously. Owing to more favorable pharmacokinetic properties, preference is nowadays more and more given to low-molecular-weight heparin, however, even with low-molecular-weight heparin, it is not possible to avoid the known disadvantages described below, which are involved in heparin therapy. Thus, heparin is ineffective when administered orally and has a relatively short half-life. Since heparin inhibits a plurality of factors of the blood coagulation cascade at the same time, the action is nonselective. Moreover, there is a high risk of bleeding; in particular, brain haemorrhages and gastrointestinal bleeding may occur, which may result in thrombopenia, drug-induced alopecia or osteoporosis [Pschyrembel, Klinisches Worterbuch, 257th edition, 1994, Walter de Gruyter Verlag, page 610, entry “Heparin”; Römpp Lexikon Chemie, Version 1.5, 1998, Georg Thieme Verlag Stuttgart, entry “Heparin”].

A second class of anticoagulants are the vitamin K antagonists. These include, for example, 1,3-indanediones, and especially compounds such as warfarin, phenprocoumon, dicumarol and other coumarin derivatives which inhibit the synthesis of various products of certain vitamin Kdependent coagulation factors in the liver in a nonselective manner. Owing to the mechanism of action, however, the onset of the action is very slow (latency to the onset of action 36 to 48 hours). It is possible to administer the compounds orally; however, owing to the high risk of bleeding and the narrow therapeutic index, a time-consuming individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683].

Recently, a novel therapeutic approach for the treatment and prophylaxis of thromboembolic disorders has been described. This novel therapeutic approach aims to inhibit factor Xa. Because of the central role which factor Xa plays in the blood coagulation cascade, factor Xa is one of the most important targets for anticoagulants [J. Hauptmann, J. Stürzebecher, Thrombosis Research 1999, 93, 203; S. A. V. Raghavan, M. Dikshit, “Recent advances in the status and targets of anti-thrombotic agents” Drugs Fut. 2002, 27, 669-683; H. A. Wieland, V. Laux, D. Kozian, M. Lorenz, “Approaches in anticoagulation: Rationales for target positioning” Curr. Opin. Investig. Drugs 2003, 4, 264-271; U. J. Ries, W. Wienen, “Serine proteases as targets for antithrombotic therapy” Drugs Fut. 2003, 28, 355-370; L.-A. Linkins, J. I. Weitz, “New anticoagulant therapy” Annu. Rev. Med. 2005, 56, 63-77 (online publication August 2004).

It has been shown that, in animal models, various both peptidic and nonpeptidic compounds are effective as factor Xa inhibitors. A large number of direct factor Xa inhibitors is already known [J. M. Walenga, W. P. Jeske, D. Hoppensteadt, J. Fareed, “Factor Xa Inhibitors: Today and beyond” Curr. Opin. Investig. Drugs 2003, 4, 272-281; J. Ruef, H. A. Katus, “New antithrombotic drugs on the horizon” Expert Opin. Investig. Drugs 2003, 12, 781-797; M. L. Quan, J. M. Smallheer, “The race to an orally active Factor Xa inhibitor: Recent advances” Curr. Opin. Drug Discovery & Development 2004, 7, 460-469]. Nonpeptidic factor Xa inhibitors having an oxazolidinone core structure are described in WO 01/047919 and WO 02/064575.

It is an object of the present invention to provide novel substances for controlling disorders, in particular thromboembolic disorders, which substances have improved solubility in water and physiological media.

The present invention provides compounds of the general formula (I)

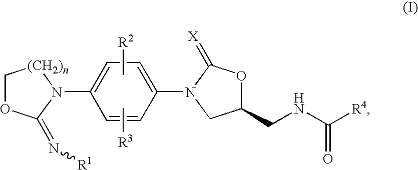

in which
R¹ represents hydrogen, hydroxyl, cyano, (C₁-C₆)-alkyl, (C₁-C₆)-alkanoyl, benzoyl or heteroaroyl,
R² and R³ are identical or different and independently of one another represent hydrogen, fluorine, chlorine, cyano, (C₁-C₄)-alkyl, cyclopropyl, trifluoromethyl, hydroxyl, (C₁-C₄)-alkoxy, trifluoromethoxy, amino, mono- or di-(C₁-C₄)-alkylamino,
R⁴ represents phenyl, pyridyl, pyrimidinyl, pyrazinyl, thienyl, furyl or pyrrolyl which may in each case be mono- or disubstituted by identical or different substituents selected from the group consisting of halogen, cyano, (C₁-C₄)-alkyl, which for its part may be substituted by hydroxyl or amino, (C₁-C₄)-alkoxy, ethynyl, cyclopropyl and amino,
n represents the number 1, 2 or 3,
   and
X represents O or N—R⁵, where
   R⁵ represents hydrogen, cyano, (C₁-C₆)-alkyl or phenyl,
      where phenyl may be mono- or disubstituted by identical or different substituents selected from the group consisting of halogen, cyano, trifluoromethyl, (C₁-C₄)alkyl and (C₁-C₄)-alkoxy,
         and salts, solvates and solvates of the salts thereof.

The compounds according to the invention are the compounds of the formula (I) and their salts, solvates and solvates of the salts, the compounds, comprised by formula (I), of the formulae mentioned below and their salts, solvates and solvates of the salts and the compounds, comprised by formula (I), mentioned below as embodiments and their salts, solvates and solvates of the salts if the compounds, comprised by formula (I), mentioned below are not already salts, solvates and solvates of the salts.

Depending on their structure, the compounds according to the invention can exist in stereoisomeric forms (enantiomers, diastereomers). Accordingly, the invention comprises the enantiomers or diastereomers and their respective mixtures. From such mixtures of enantiomers and/or diastereomers, it is possible to isolate the stereoisomerically uniform components in a known manner.

If the compounds according to the invention can be present in tautomeric forms, the present invention comprises all tautomeric forms.

In the context of the present invention, preferred salts are physiologically acceptable salts of the compounds according to the invention. The invention also comprises salts which for their part are not suitable for pharmaceutical applications, but which can be used, for example, for isolating or purifying the compounds according to the invention.

Physiologically acceptable salts of the compounds according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalene disulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the compounds according to the invention which, in solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the compounds according to the invention. The term “prodrugs” includes compounds which for their part may be biologically active or inactive but which, during the time they spend in the body, are converted into compounds according to the invention (for example metabolically or hydrolytically).

In the context of the present invention, unless specified differently, the substituents have the following meanings:

In the context of the invention, (C₁-C₆)-alkyl and (C₁-C₄)alkyl represent a straight-chain or branched alkyl radical having 1 to 6 and 1 to 4 carbon atoms, respectively. Preference is given to a straight-chain or branched alkyl radical having 1 to 4 carbon atoms. The following radicals may be mentioned by way of example and by way of preference: methyl, ethyl, n-propyl, isopropyl, n-butyl, isobutyl, sec-butyl, tert-butyl, 1-ethylpropyl, n-pentyl and n-hexyl.

In the context of the invention, (C₁-C₄)-alkoxy represents a straight-chain or branched alkoxy radical having 1 to 4 carbon atoms. The following radicals may be mentioned by way of example and by way of preference: methoxy, ethoxy, n-propoxy, isopropoxy, n-butoxy and tert-butoxy.

In the context of the invention, (C₁-C₆)-alkanoyl and [(C₁-C₆)-acyl] represents a straight-chain or branched alkyl radical having 1 to 6 carbon atoms which carries a doubly attached oxygen atom in the 1-position and is attached via the 1-position. Preference is given to a straight-chain or branched alkanoyl radical having 1 to 4 carbon atoms. The following radicals may be mentioned by way of example and by way of preference: formyl, acetyl, propionyl, n-butyryl, isobutyryl and pivaloyl.

In the context of the invention, mono-(C₁-C₄)-alkylamino represent an amino group having a straight-chain or branched alkyl substituent having 1 to 4 carbon atoms. The following radicals may be mentioned by way of example and by way of preference: methylamino, ethylamino, npropylamino, isopropylamino, n-butylamino and tert-butylamino.

In the context of the invention, di-(C₁-C₄)-alkylamino represents an amino group having two identical or different straight-chain or branched alkyl substituents having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, Nisopropyl-N-methylamino, N-isopropyl-N-n-propylamino, N-n-butyl-N-methylamino and N-tertbutyl-N-methylamino.

In the context of the invention, heteroaroyl (heteroarylcarbonyl) represents an aromatic heterocycle (heteroaromatic) having a total of 5 or 6 ring atoms and up to three identical or different ring heteroatoms from the group consisting of N, O and S, which is attached via a carbonyl group. The following radicals may be mentioned by way of example: furoyl, pyrroyl, thienoyl, pyrazoyl, imidazoyl, thiazoyl, oxazoyl, isoxazoyl, isothiazoyl, triazoyl, oxadiazoyl, thiadiazoyl, pyridinoyl, pyrimidinoyl, pyridazinoyl, pyrazinoyl. Preference is given to a 5- or 6-membered heteroaroyl radical having up to two heteroatoms from the group consisting of N, O and S, such as, for example, furoyl, thienoyl, thiazoyl, oxazoyl, isoxazoyl, isothiazoyl, pyridinoyl, pyrimidinoyl, pyridazinoyl, pyrazinoyl.

In the context of the invention, halogen includes fluorine, chlorine, bromine and iodine. Preference is given to fluorine or chlorine.

If radicals in the compounds according to the invention are substituted, the radicals can, unless specified otherwise, be mono- or polysubstituted. In the context of the present invention, the meanings of radicals which occur more than once are independent of one another. Substitution with one, two or three identical or different substituents is preferred. Very particular preference is given to substitution with one substituent.

Preference is given to compounds of the formula (I) in which
R¹ represents hydrogen, hydroxyl, cyano, (C₁-C₆)-alkyl, (C₁-C₆)-alkanoyl, benzoyl or heteroaroyl,
R² and R³ are identical or different and independently of one another represent hydrogen, fluorine, chlorine, cyano, (C₁-C₄)-alkyl, cyclopropyl, trifluoromethyl, hydroxyl, (C₁-C₄)-alkoxy, trifluoromethoxy, amino, mono- or di-(C₁-C₄)-alkylamino,
R⁴ represents phenyl, pyridyl, pyrimidinyl, pyrazinyl, thienyl, furyl or pyrrolyl which may in each case be mono- or disubstituted by identical or different substituents selected from the group consisting of halogen, cyano, (C₁-C₄)-alkyl, which for its part may be substituted by hydroxyl or amino, (C₁-C₄)-alkoxy, ethynyl, cyclopropyl and amino,
n represents the number 1, 2 or 3,
   and
X represents O or N—R⁵, where
   R⁵ represents hydrogen, cyano or (C₁-C₆)-alkyl,
      and salts, solvates and solvates of the salts thereof.

Particular preference is given to compounds of the formula (I) in which

R¹ represents hydrogen, hydroxyl, cyano or methyl,

R² represents hydrogen,

R³ represents hydrogen, fluorine, chlorine, cyano, methyl or methoxy,

R⁴ represents a group of the formula

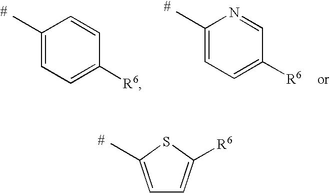

in which
R⁶ represents fluorine, chlorine, methyl or ethynyl
and
# denotes the point of attachment to the carbonyl group,

n represents the number 1 or 2,

and

X represents O,

and salts, solvates and solvates of the salts thereof.

Very particular preference is given to compounds of the formula (I) in which

R¹ represents hydrogen,

R² represents hydrogen,

R³ represents hydrogen, fluorine or methyl,

R⁴ represents a group of the formula

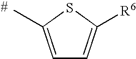

in which
R⁶ represents fluorine, chlorine or methyl
and
# denotes the point of attachment to the carbonyl group,

n represents the number 1 or 2,

and

X represents O,

and salts, solvates and solvates of the salts thereof.

Preference is also given to compounds of the formula (I) in which R¹ represents hydrogen.

Preference is also given to compounds of the formula (I) in which R² and R³ represent hydrogen.

Preference is also given to compounds of the formula (I) in which

R⁴ represents a group of the formula

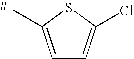

where # denotes the point of attachment to the carbonyl group.

Preference is also given to compounds of the formula (I) in which X represents O.

Independently of the respective given combinations of the radicals, the specific radical definitions given in the respective combinations or preferred combinations of radicals may also be replaced by radical definitions of other combinations.

Very particular preference is given to combinations of two or more of the preferred ranges mentioned above.

The invention furthermore provides a process for preparing the compounds of the formula (I) according to the invention in which R¹ represents hydrogen and X represents oxygen, characterized in that compounds of the formula (II)

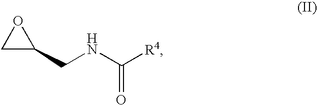

in which R⁴ has the meaning given above,
are reacted in an inert solvent, if appropriate in the presence of a Lewis acid, with a compound of the formula (III)

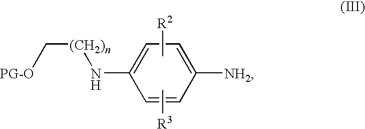

in which n, R² and R³ have the meanings given above
and
PG represents a hydroxyl protective group, preferably trimethylsilyl or tert.-butyldimethylsilyl, to give compounds of the formula (IV)

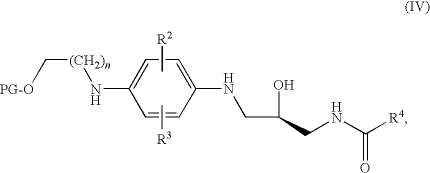

in which n, PG, R², R³ and R⁴ have the meanings given above,
these are then reacted in an inert solvent with a carbonic acid equivalent, for example N,N′-carbonyldiimidazole, to give compounds of the formula (V)

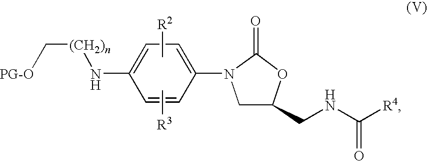

in which n, PG, R², R³ and R⁴ have the meanings given above,
and then either
[A] by removal of the protective group PG under customary conditions converted into compounds of the formula (VI)

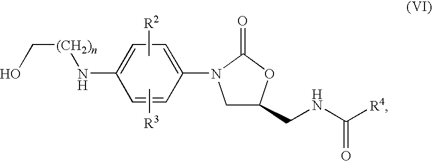

in which n, R², R³ and R⁴ have the meanings given above,
and the compounds of the formula (VI) are then in an inert solvent in the presence of an acid converted with cyanogen bromide into compounds of the formula (I-A)

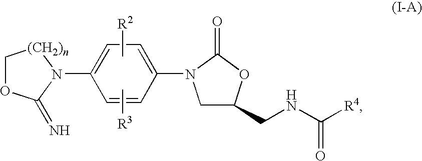

in which n, R², R³ and R⁴ have the meanings given above,
   or
[B] initially reacted in an inert solvent with cyanogen bromide, preferably in the presence of a base, to give compounds of the formula (VII)

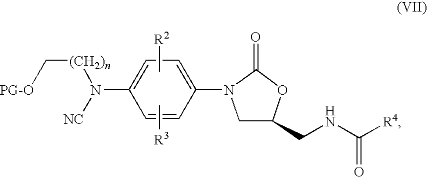

in which n, PG, R², R³ and R⁴ have the meanings given above,
then by removal of the protective group PG converted into compounds of the formula (VIII)

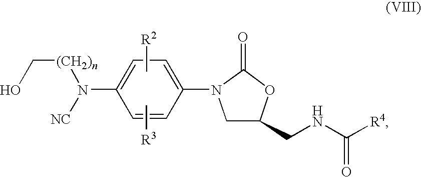

in which n, R², R³ and R⁴ have the meanings given above,
and the compounds of the formula (VIII) are then cyclized in an inert solvent in the presence of an acid to give compounds of the formula (I-A)
   and the compounds of the formula (I-A) are, if appropriate, converted with the appropriate (i) solvents and/or (ii) bases or acids into their solvates, salts and/or solvates of the salts.

The invention furthermore provides a process for preparing the compounds of the formula (I) according to the invention in which R¹ represents hydrogen and X represents NH, characterized in that, starting with compounds of the formula (IV), initially by again introducing a hydroxyl protective group PG, compounds of the formula (IX)

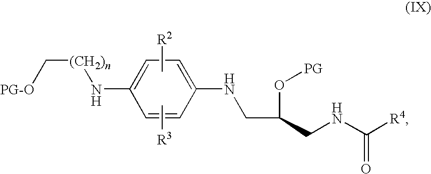

in which n, PG, R², R³ and R⁴ have the meanings given above,
are prepared, which are then, in an inert solvent using cyanogen bromide, preferably in the presence of a base, converted into compounds of the formula (X)

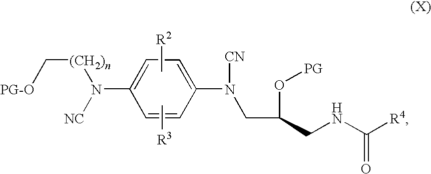

in which n, PG, R², R³ and R⁴ have the meanings given above,
then, by removing the protective groups PG, converted into compounds of the formula (XI)

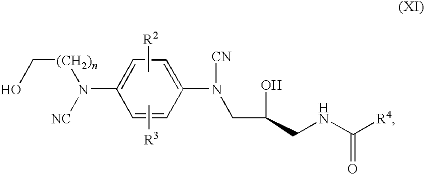

in which n, R², R³ and R⁴ have the meanings given above,
and these are cyclized in an inert solvent in the presence of an acid to compounds of the formula (I-B)

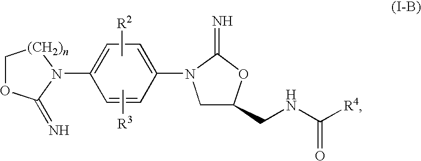

in which n, R², R³ and R⁴ have the meanings given above,
and the compounds of the formula (I-B) are, if appropriate, converted with the appropriate (i) solvents and/or (ii) bases or acids into their solvates, salts and/or solvates of the salts.

The compounds of the formula (I) according to the invention in which R¹ does not represent hydrogen can be prepared from the compounds of the formula (VI) analogously to processes known from the literature [cf., for example, for R¹=alkanoyl: D. Douglass, J. Amer. Chem. Soc. 1934, 56, 719 and T. Shibanuma, M. Shiono, T. Mukaiyama, Chem. Lett. 1977, 575-576; for R¹=cyano: a) R. Evers, M. Michalik, J. Prakt. Chem. 1991, 333, 699-710; N. Maezaki, A. Furusawa, S. Uchida, T. Tanaka, Tetrahedron 2001, 57, 9309-9316; G. Berecz, J. Reiter, G. Argay, A. Kalman, J Heterocycl. Chem. 2002, 39, 319-326; b) R. Mohr, A. Buschauer, W. Schunack, Arch. Pharm. (Weinheim Ger.) 1988, 321, 221-227; for R¹=alkyl: a) V. A. Vaillancourt et al., J. Med. Chem. 2001, 44, 1231-1248; b) F. B. Dains et al., J. Amer. Chem. Soc. 1925, 47, 1981-1989; J. Amer. Chem. Soc. 1922, 44, 2637-2643 and T. Shibanuma, M. Shiono, T. Mukaiyama, Chem. Lett. 1977, 575-576; see also Synthesis Schemes 3 and 4].

If appropriate, the compounds according to the invention can also be prepared by further conversions of functional groups of individual substituents, in particular those listed for R², R³ and R⁴, starting from the compounds of the formula (I) obtained by the above processes. These conversions are carried out by customary methods and include, for example, reactions such as alkylation, amination, acylation, esterification, ester cleavage, amide formation, oxidation or reduction, and also the introduction and removal of temporary protective groups.

Inert solvents for the process step (II)+(II)→(IV) are, for example, alcohols, such as methanol, ethanol, n-propanol, isopropanol, n-butanol or tert-butanol, ethers, such as diethyl ether, dioxane, tetrahydrofuran, 2-methyltetrahydrofuran, glycol dimethyl ether or diethylene glycol dimethyl ether, or other solvents, such as acetone, dimethylformamide, dimethyl sulfoxide, N,N′-dimethylpropyleneurea (DMPU), N-methylpyrrolidone (NMP), acetonitrile or else water. It is also possible to use mixtures of the solvents mentioned. Preference is given to using dioxane, tetrahydrofuran, ethanol or mixtures thereof with water.

If appropriate, the process step (II)+(III)→(IV) can also be carried out with addition of a catalytic amount of a Lewis acid, such as, for example, ytterbium (II) trifluoromethanesulfonate.

The reaction (II)+(III)→(IV) is generally carried out in a temperature range of from 0° C. to +100° C., preferably from +20° C. to +80° C.

The ring closure to the oxazolidinone in process step (IV)→(V) is preferably carried out using N,N′-carbonyldiimidazole as carbonic acid equivalent with addition of 4-N,N-dimethylaminopyridine as base. The reaction is preferably carried out in tetrahydrofuran as solvent in a temperature range of from +20° C. to +70° C.

In the process steps (V)→(VI), (VII)→(VIII) and (X)→(XI), the removal of trimethylsilyl or tert-butyldimethylsilyl as preferred hydroxyl protective groups (PG) can preferably be carried out with the aid of tetra-n-butylammonium fluoride (TBAF) or, in the case of the reaction (V)→(VI), also with hydrogen fluoride. The reactions are generally carried out in tetrahydrofuran as solvent in a temperature range of from 0° C. to +40° C.

With particular preference, the reaction sequences (VII)→(VIII)→(I-A) and (X)→(XI)→(I-B) in total are carried out using an acid-labile hydroxyl protective group, such as, for example, trimethylsilyl or tert-butyldimethylsilyl, in the presence of an excess of an acid as a one-pot reaction, without isolation of the intermediate (VIII) and (XI), respectively.

Suitable inert solvents for the process steps (VI)→(I-A), (V)→(VII), (VIII)→(I-A), (IX)→(X) and (XI)→(I-B) are in particular tetrahydrofuran, dichloromethane, acetonitrile or mixtures of these solvents. These process steps are generally carried out in a temperature range of from −20° C. to +50° C., preferably from 0° C. to +40° C.

Suitable acids for the process steps (VI)→(I-A) and (VIII)→(I-A) and the reaction sequences (VII)→(VIII)→(I-A) and (X)→(XI)→(I-B) are in particular strong inorganic or organic acids, such as, for example, hydrogen fluoride, hydrogen chloride, hydrogen bromide, methanesulfonic acid, trifluoromethanesulfonic acid or trifluoroacetic acid.

The process steps (V)→(VII) and (IX)→(X) are preferably carried out in the presence of a base. Suitable for this purpose are in particular inorganic bases, such as, for example, alkali metal or alkaline earth metal carbonates or bicarbonates, such as lithium carbonate, sodium carbonate, potassium carbonate, calcium carbonate or cesium carbonate or sodium bicarbonate or potassium bicarbonate, or alkali metal hydrides, such as sodium hydride.

The reactions mentioned can be carried out under atmospheric, elevated or reduced pressure (for example at from 0.5 to 5 bar). In general, the reactions are in each case carried out under atmospheric pressure.

The compounds of the formula (II) can be prepared by the process described in WO 2004/101557 from (2S)-3-aminopropane-1,2-diol of the formula (XII)

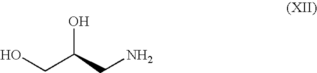

and carboxylic acid derivatives of the formula (XII)

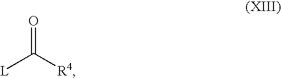

in which R⁴ has the meaning given above and

L represents a leaving group, such as, for example, halogen, in particular chlorine.

The compounds of the formula (III) can be obtained analogously to processes known from the literature, for example by reacting compounds of the formula (XIV)

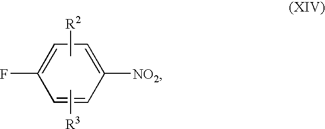

in which R² and R³ have the meanings given above,
with a compound of the formula (XV)

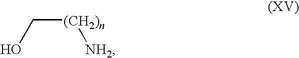

in which n has the meaning given above,
to give compounds of the formula (XVI)

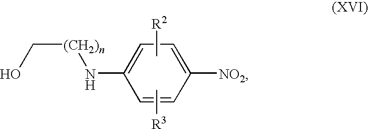

in which n, R² and R³ have the meanings given above,
subsequent introduction of the hydroxyl protective group PG and then reduction of the nitro group to the amine.

The compounds of the formulae (XII), (XIII), (XIV) and (XV) are commercially available, known from the literature or can be prepared analogously to processes known from the literature.

The preparation of the compounds according to the invention can be illustrated by the synthesis schemes below:

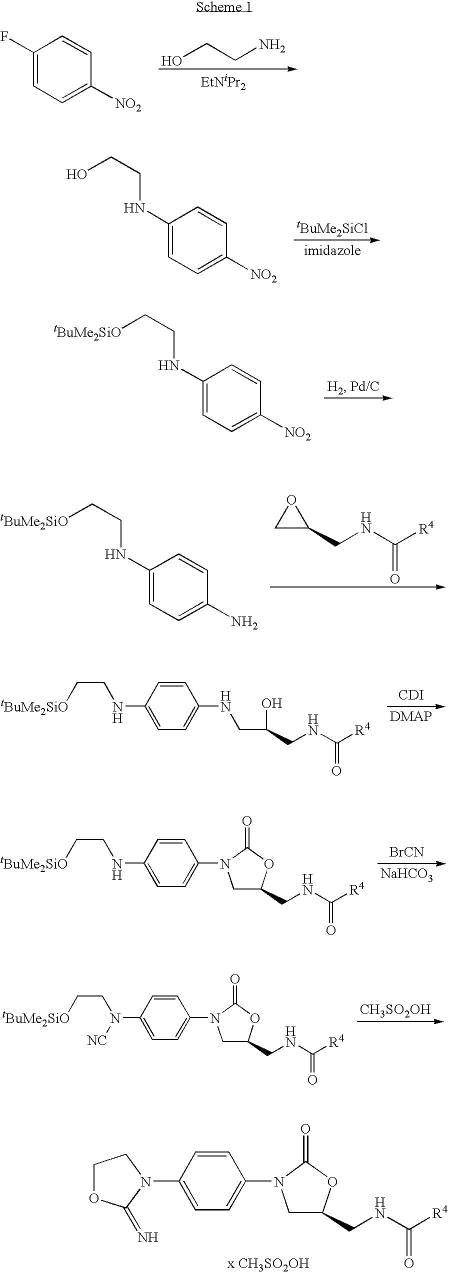

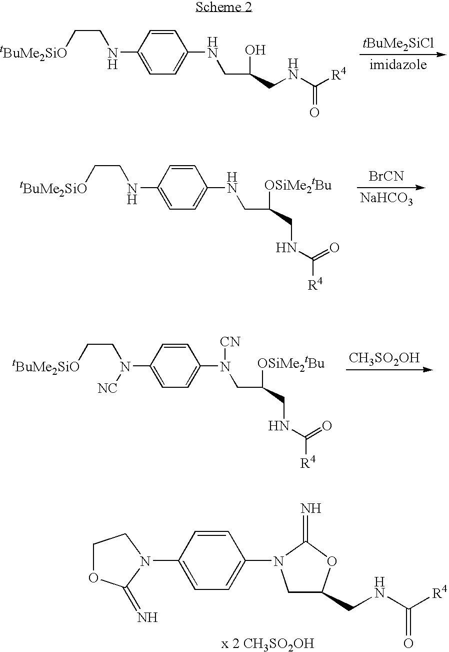

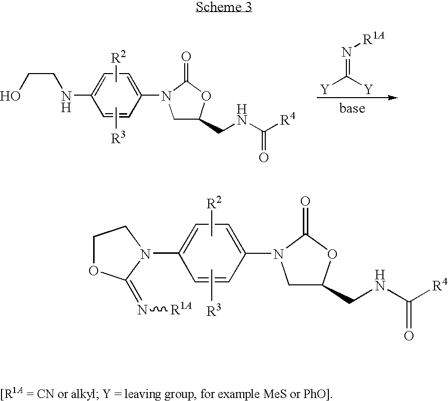

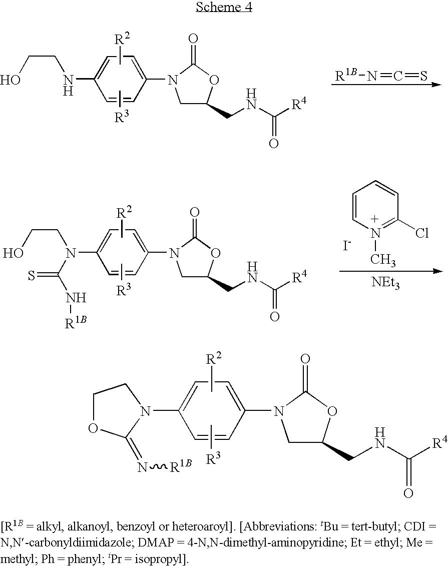

The compounds according to the invention have an unforeseeable useful pharmacological activity spectrum.

Accordingly, they are suitable for use as medicaments for the treatment and/or prophylaxis of diseases in humans and animals.

The compounds according to the invention are selective inhibitors of blood coagulation factor Xa which act in particular as anticoagulants.

In addition, the compounds according to the invention have favorable physicochemical properties, such as, for example, good solubility in water and physiological media, which is advantageous for their therapeutic application.

The present invention furthermore provides the use of the compounds according to the invention for the treatment and/or prophylaxis of disorders, preferably thromboembolic disorders and/or thromboembolic complications.

For the purposes of the present invention, “thromboembolic disorders” include in particular disorders such as ST-elevation myocardial infarction (STEMI) or non-ST-elevation myocardial infarction (non-STEMI), stable angina pectoris, unstable angina pectoris, reocclusions and restenoses after coronary interventions such as angioplasty or aortocoronary bypass, peripheral arterial occlusive diseases, pulmonary embolisms, deep vein thromboses and kidney vein thromboses, transitory ischaemic attacks and also thrombotic and thromboembolic stroke.

Accordingly, the substances are also suitable for preventing and treating cardiogenic thromboembolisms, such as, for example, brain ischaemias, stroke and systemic thromboembolisms and ischaemias, in patients having acute, intermittent or persistent cardioarrhythmias, such as, for example, atrial fibrillation, and those undergoing cardioversion, furthermore patients having heart valve disorders or having artificial heart valves. In addition, the compounds according to the invention are suitable for treating disseminated intravascular coagulation (DIC).

Thromboembolic complications furthermore occur during microangiopathic haemolytic anaemias, extracorporeal blood circulation, such as haemodialysis, and in connection with heart valve prostheses.

Moreover, the compounds according to the invention are also suitable for the prophylaxis and/or treatment of atherosclerotic vascular disorders and inflammatory disorders, such as rheumatic disorders of the locomotor apparatus, and in addition also for the prophylaxis and/or treatment of Alzheimer's disease. Moreover, the compounds according to the invention can be used for inhibiting tumor growth and formation of metastases, for microangiopathies, age-related macular degeneration, diabetic retinopathy, diabetic nephropathy and other microvascular disorders, and also for the prevention and treatment of thromboembolic complications, such as, for example, venous thromboembolisms, in tumor patients, in particular patients undergoing major surgical interventions or chemo- or radiotherapy.

The compounds according to the invention can additionally also be used for preventing coagulation ex vivo, for example for preserving blood and plasma products, for cleaning/pretreating catheters and other medical tools and instruments, for coating synthetic surfaces of medical tools and instruments used in vivo or ex vivo or for biological samples comprising factor Xa.

The present invention furthermore provides the use of the compounds according to the invention for the treatment and/or prophylaxis of disorders, in particular the disorders mentioned above.

The present invention furthermore provides the use of the compounds according to the invention for preparing a medicament for the treatment and/or prophylaxis of disorders, in particular the disorders mentioned above.

The present invention furthermore provides a method for the treatment and/or prophylaxis of disorders, in particular the disorders mentioned above, using an anticoagulatorilly effective amount of the compound according to the invention.

The present invention furthermore provides a method for preventing blood coagulation in vitro, in particular in banked blood or biological samples comprising factor Xa, which method is characterized in that an anticoagulatorilly effective amount of the compound according to the invention is added.

The present invention furthermore provides medicaments comprising a compound according to the invention and one or more further active compounds, in particular for the treatment and/or prophylaxis of the disorders mentioned above. The following compounds may be mentioned by way of example and by way of preference as active compounds suitable for combinations:
lipid-lowering agents, in particular HMG-CoA (3-hydroxy-3-methylglutaryl-coenzyme A) reductase inhibitors;
coronary therapeutics/vasodilators, in particular ACE (angiotensin converting enzyme) inhibitors; AII (angiotensin II) receptor antagonists; β-adrenoceptor antagonists; alpha-1-adrenoceptor antagonists; diuretics; calcium channel blockers; substances which cause an increase in the cyclic guanosine monophosphate (cGMP) concentration such as, for example, stimulators of soluble guanylate cyclase;
plasminogen activators (thrombolytics/fibrinolytics) and compounds enhancing thrombolysis/fibrinolysis, such as inhibitors of the plasminogen activator inhibitor (PAI inhibitors) or inhibitors of the thrombin-activated fibrinolysis inhibitor (TAFI inhibitors);
anticoagulants;
platelet aggregation inhibiting substances (platelet aggregation inhibitors, thrombocyte aggregation inhibitors);
fibrinogen receptor antagonists (glycoprotein-IIb/IIIa antagonists);
and also antiarrhythmics.

The present invention furthermore provides medicaments comprising at least one compound according to the invention, usually together with one or more inert non-toxic, pharmaceutically acceptable auxiliaries, and their use for the purposes mentioned above.

The compounds according to the invention can act systemically and/or locally. For this purpose, they can be administered in a suitable way, such as, for example, by the oral, parenteral, pulmonary, nasal, sublingual, lingual, buccal, rectal, dermal, transdermal, conjunctival or otic route, or as implant or stent.

For these administration routes, it is possible to administer the compounds according to the invention in suitable administration forms.

Suitable for oral administration are administration forms which work as described in the prior art and deliver the compounds according to the invention rapidly and/or in modified form, which comprise the compounds according to the invention in crystalline and/or amorphous and/or dissolved form, such as, for example, tablets (uncoated and coated tablets, for example tablets provided with enteric coatings or coatings whose dissolution is delayed or which are insoluble and which control the release of the compound according to the invention), tablets which rapidly decompose in the oral cavity, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (for example intravenously, intraarterially, intracardially, intraspinally or intralumbarly) or with inclusion of absorption (for example intramuscularly, subcutaneously, intracutaneously, percutaneously or intraperitoneally). Administration forms suitable for parenteral administration are, inter alia, preparations for injection and infusion in the form of solutions, suspensions, emulsions, lyophilizates or sterile powders.

Examples suitable for the other administration routes are pharmaceutical forms for inhalation (inter alia powder inhalers, nebulizers), nasal drops/solutions/sprays, tablets to be administered lingually, sublingually or buccally, films/wafers or capsules, suppositories, preparations for the eyes or ears, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Preference is given to oral or parenteral administration, in particular oral administration.

The compounds according to the invention can be converted into the stated administration forms. This can take place in a manner known per se by mixing with inert, nontoxic, pharmaceutically suitable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides) and flavor- and/or odor-masking agents.

In general, it has proved advantageous to administer on parenteral administration amounts of from about 0.001 to 1 mg/kg, preferably from about 0.01 to 0.5 mg/kg, of body weight to achieve effective results. The dosage on oral administration is from about 0.01 to 100 mg/kg, preferably about 0.01 to 20 mg/kg, and very particularly preferably 0.1 to 10 mg/kg, of body weight.

It may nevertheless be necessary, where appropriate, to deviate from the amounts mentioned, depending on the body weight, the administration route, the individual response to the active compound, the mode of preparation and the time or interval over which administration takes place. Thus, in some cases it may be sufficient to make do with less than the aforementioned minimal amount, whereas in other cases the upper limit mentioned must be exceeded. In the event of administration of larger amounts, it may be advisable to divide these into a plurality of individual doses over the day.

The invention is illustrated by the working examples below. The invention is not limited to the examples.

The percentage data in the following tests and examples are percentages by weight unless otherwise indicated; parts are parts by weight. Solvent ratios, dilution ratios and concentration data of liquid/liquid solutions are in each case based on volume.

### A. EXAMPLES

### Abbreviations and Acronyms

d day(s)

DMF N,N-dimethylformamide

DMSO dimethyl sulfoxide

eq. equivalent(s)

ESI electrospray ionization (in MS)

h hour(s)

HPLC high pressure, high performance liquid chromatography

LC-MS liquid chromatography-coupled mass spectroscopy

min minute(s)

MS mass spectroscopy

NMR nuclear magnetic resonance spectroscopy

RP reverse phase (in HPLC)

Rₜ retention time (in HPLC)

RT room temperature

THF tetrahydrofuran

### LC-MS and HPLC Methods:

### Method 1:

MS instrument type: Micromass ZQ; HPLC instrument type: Waters Alliance 2795; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 2:

MS instrument type: Micromass ZQ; HPLC instrument type: HP 1100 Series; UV DAD; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 3:

Instrument: Micromass Quattro LCZ with HPLC Agilent Series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 208-400 nm.

### Method 4:

Instrument: Micromass Platform LCZ with HPLC Agilent Series 1100; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 5:

Instrument: Micromass Platform LCZ with HPLC Agilent Series 1100; column: Thermo HyPURITY Aquastar 3μ 50 mm×2.1 mm; mobile phase A: 1 l of water+0.5 ml of 50% strength formic acid, mobile phase B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm.

### Method 6:

MS instrument type: Micromass ZQ; HPLC instrument type: Waters Alliance 2795; column: Merck Chromolith SpeedROD RP-18e 50 mm×4.6 mm; mobile phase A: 1 l of water+0.5 ml of 50% strength formic acid, mobile phase B: 1 l of acetonitrile+0.5 ml of 50% strength formic acid; gradient: 0.0 min 10% B→3.0 min 95% B→4.0 min 95% B; oven: 35° C.; flow rate: 0.0 min 1.0 ml/min→3.0 min 3.0 ml/min→4.0 min→3.0 ml/min; UV detection: 210 nm.

### Method 7:

Instrument: HP 1100 with DAD detection; column: Kromasil 100 RP-18, 60 mm×2.1 mm, 3.5 μm; mobile phase A: 5 ml of HClO₄ (70% strength)/1 of water, mobile phase B: acetonitrile; gradient: 0 min 2% B→0.5 min 2% B→4.5 min 90% B→9 min 90% B<9.2 min 2% B→10 min 2% B; flow rate: 0.75 ml/min; column temperature: 30° C.; UV detection: 210 nm.

### Method 8:

Instrument: HP 1100 with DAD detection; column: Kromasil 100 RP-18, 60 mm×2.1 mm, 3.5 μm; mobile phase A: 5 ml of HClO₄ (70% strength)/1 of water, mobile phase B: acetonitrile; gradient: 0 min 2% B→0.5 min 2% B→4.5 min 90% B→15 min 90% B→15.2 min 2% B→16 min 2% B; flow rate: 0.75 ml/min; column temperature: 30° C.; UV detection: 210 nm.

### Method 9:

Instrument: HP 1100 with DAD detection; column: Kromasil 100 RP-18, 60 mm×2.1 mm, 3.5 μm; mobile phase A: 5 ml of HClO₄ (70% strength)/1 of water, mobile phase B: acetonitrile; gradient: 0 min 2% B→0.5 min 2% B→4.5 min 90% B→6.5 min 90% B→6.7 min 2% B 7.5 min 2% B; flow rate: 0.75 ml/min; column temperature: 30° C.; UV detection: 210 nm n.

### Starting Materials and Intermediates

### Example 1A

### N-(2-{[tert-Butyl(dimethyl)silyl]oxy}ethyl)benzene-1,4-diamine

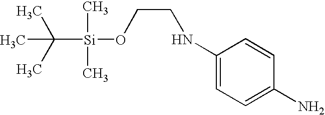

### Step a): 2-[(4-Nitrophenyl)amino]ethanol

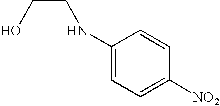

130 ml (2.15 mol, 3 eq.) of 2-aminoethanol and 274 ml (1.57 mol, 2.2 eq.) of N,N-diisopropylethylamine are added to a solution of 10 g (716 mmol) of 4-fluoronitrophenol in 500 ml of ethanol. The reaction mixture is stirred at 50° C. overnight, a further 86 ml (1.43 mol, 2.0 eq.) of 2-aminoethanol and 249 ml (1.43 mol, 2.0 eq.) of N,N-diisopropylethylamine are added and the mixture is again stirred at 50° C. for 12 h. The reaction solution is then concentrated under reduced pressure, and the residue is triturated with 600 ml of water. The resulting precipitate is filtered off, washed repeatedly with water and dried.

Yield: 127 g (97% of theory)

LC-MS (method 5): Rₜ=2.32 min;

MS (ESIpos): m/z=183 [M+H]⁺;

¹H-NMR (300 MHz, DMSO-d₆): δ=7.99 (d, 2H), 7.30 (t, 1H), 6.68 (d, 2H), 4.82 (t, 1H), 3.63-3.52 (m, 2H), 3.30-3.19 (m, 2H).

### Step b): N-(2-{[tert-Butyl(dimethyl)silyl]oxy}ethyl)-4-nitroaniline

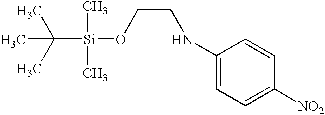

At room temperature, 30.6 g (203 mmol, 1.2 eq.) of tert-butyldimethylchlorosilane and 17.3 g (254 mmol, 1.5 eq.) of imidazole are added to a solution of 30.8 g (169 mmol) of 2-[(4-nitrophenyl)amino]ethanol in 300 ml DMF, and the mixture is stirred at RT for 2.5 h. The reaction mixture is concentrated under reduced pressure, and the residue is dissolved in 200 ml of dichloromethane and 100 ml of water. After phase separation, the aqueous phase is extracted three times with in each case 80 ml of dichloromethane. The combined organic phases are washed with 100 ml of saturated aqueous sodium chloride solution, dried over sodium sulfate, filtered and concentrated under reduced pressure.

Yield: 49.7 g (quant.)

LC-MS (method 3): Rₜ=3.09 min;

MS (ESIpos): m/z=297 [M+H]⁺;

¹H-NMR (300 MHz, DMSO-d₆): δ=7.98 (d, 2H), 7.29 (t, 1H), 6.68 (d, 2H), 3.77-3.66 (m, 2H), 3.35-3.24 (m, 2H), 0.81 (s, 9H), 0.0 (s, 6H).

### Step c): N-(2-{[tert-Butyl(dimethyl)silyl]oxy}ethyl)benzene-1,4-diamine

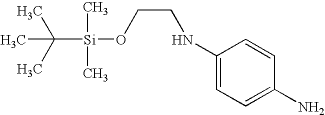

Under argon, 4 g of palladium-on-carbon (10%) are added to a solution of 59.5 g (201 mmol) of N-(2-{[tert-butyl(dimethyl)silyl]oxy}ethyl)-4-nitroaniline in 500 ml of ethanol, and the mixture is hydrogenated in a hydrogen atmosphere at RT and under atmospheric pressure. The catalyst is separated off through a filter layer and washed with ethanol, and the filtrate is concentrated under reduced pressure.

Yield: 53 g (quant.)

LC-MS (method 2): Rₜ=1.83 min;

MS (ESIpos): m/z=267 [M+H]⁺;

¹H-NMR (300 MHz, DMSO-d₆): δ=6.42-6.30 (m, 4H), 4.48 (t, 1H), 4.21 (br. s, 2H), 3.68-3.58 (m, 2H), 3.04-2.93 (m, 2H), 0.82 (s, 9H), 0.0 (s, 6H).

### Example 2A

### N-(3-{[tert-Butyl(dimethyl)silyl]oxy}propyl)benzene-1,4-diamine

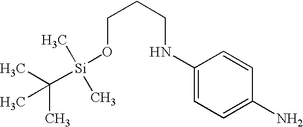

The title compound is prepared by a reaction sequence analogous to that described in Example 1A.

LC-MS (method 6): Rₜ=1.73 min;

MS (ESIpos): m/z=281 [M+H]⁺;

¹H-NMR (400 MHz, DMSO-d₆): δ=6.39 (d, 2H), 6.30 (d, 2H), 4.56 (br. s, 1H), 4.19 (br. s, 2H), 3.69-3.60 (m, 2H), 2.97-2.88 (m, 2H), 1.70-1.60 (m, 2H), 0.83 (s, 9H), 0.0 (s, 6H).

### Example 3A

### 5-Chloro-N-[(2S)-2-oxiranylmethyl]-2-thiophenecarboxamide

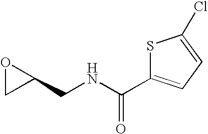

The title compound is prepared as described in WO 2004/101557 (Example 6A) by (i) acylation of (2S)-3-aminopropane-1,2-diol hydrochloride with 5-chlorothiophene-2-carbonyl chloride in the presence of sodium bicarbonate as base, (ii) hydroxyl-bromine exchange with the aid of hydrobromic acid in acetic acid/acetic anhydride and (iii) epoxide formation in the presence of potassium carbonate as base.

### Working Examples

### Example 1

### 5-Chloro-N-({(5S)-3-[4-(2-imino-1,3-oxazolidin-3-yl)phenyl]-2-oxo-1,3-oxazolidin-5-yl}methyl)thiophene-2-carboxamide

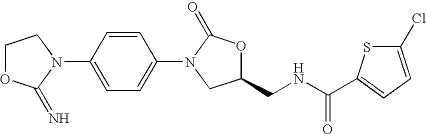

### Step a): N-[(2R)-3-({4-[(2-{[tert-Butyl(dimethyl)silyl]oxy}ethyl)amino]phenyl}amino)-2-hydroxypropyl]-5-chlorothiophene-2-carboxamide

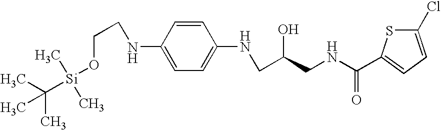

Under argon and at room temperature, 110 mg (0.17 mmol, 0.01 eq.) of ytterbium (III) trifluoromethanesulfonate are added to a solution of 4.6 g (17.3 mmol) of the compound from Example 1A in 50 ml of THF. Over a period of 2 h and at 60° C., a solution of 2.6 g (12.1 mmol, 0.7 eq.) of the compound from Example 3A in 20 ml of THF is then added dropwise to the reaction mixture. The mixture is stirred at 60° C. overnight, over a period of 1 h, another solution of 1.1 g (5.2 mmol, 0.3 eq.) of the compound from Example 3A in 10 ml of THF is added dropwise and the mixture is stirred for a further 2 h at 60° C. The reaction mixture is then concentrated under reduced pressure, and the crude product is purified by flash chromatography on silica gel (mobile phase: dichloromethane/methanol 200:1→40:1).

Yield: 3.7 g (93% purity, 41% of theory)

LC-MS (method 2): Rₜ₁=2.08 min;

MS (ESI): m/z=484 [M+H]⁺.

### Step b): N-[((5S)-3-{4-[(2-{[tert-Butyl(dimethyl)silyl]oxy}ethyl)amino]phenyl}-2-oxo-1,3-oxazolidin-5-yl)methyl]-5-chlorothiophene-2-carboxamide

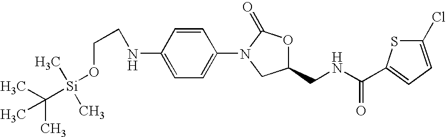

Under argon and at room temperature, 400 mg (3.30 mmol, 0.5 eq.) of 4-N,N-dimethylaminopyridine and 1.6 g (9.9 mmol, 1.4 eq.) of N,N′-carbonyldiimidazole are added to a solution of 3.7 g (93% purity, 7.1 mmol) of N-[(2R)-3-({4-[(2-{[tert-butyl(dimethyl)silyl]oxy}ethyl)amino]phenyl}-amino)-2-hydroxypropyl]-5-chlorothiophene-2-carboxamide in 110 ml of THF. The reaction mixture is stirred at room temperature overnight and then concentrated under reduced pressure. The title compound is isolated by flash chromatography of the crude product on silica gel (mobile phase: dichloromethane/methanol 80:1).

Yield: 3.6 g (94% purity, 99% of theory)

LC-MS (method 1): Rₜ, =2.81 min;

MS (ESI): m/z=510 [M+H]⁺;

¹H-NMR (300 MHz, DMSO-d₆): δ=8.93 (t, 1H), 7.66 (d, 1H), 7.17 (d, 2H), 7.15 (d, 1H), 6.56 (d, 2H), 5.43 (t, 1H), 4.78-4.67 (m, 1H), 4.03 (t, 1H), 3.70 (dd, 1H), 3.66 (t, 2H), 3.54 (t, 2H), 3.10 (qd, 2H), 0.83 (s, 9H), 0.00 (s, 6H).

### Step c): N-[((5S)-3-{4-[(2-{[tert-Butyl(dimethyl)silyl]oxy}ethyl)(cyano)amino]phenyl}-2-oxo-1,3-oxazolidin-5-yl)methyl]-5-chlorothiophene-2-carboxamide

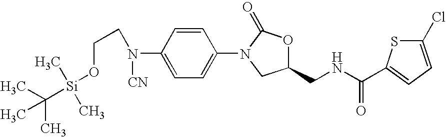

Under argon and at room temperature, 4.4 g (51.8 mmol, 3 eq.) of sodium bicarbonate and 6.9 ml of cyanogen bromide solution (3 M in dichloromethane, 20.7 mmol, 1.2 eq.) are added to a solution of 9.3 g (17.3 mmol) of N-[((5S)-3-{4-[(2-{[tert-butyl(dimethyl)silyl]oxy}ethyl)amino]-phenyl}-2-oxo-1,3-oxazolidin-5-yl)methyl]-5-chlorothiophene-2-carboxamide in 48 ml of THF. The reaction mixture is stirred at 40° C. for 2 d, and water and dichloromethane are then added. After phase separation, the organic phase is washed with saturated aqueous sodium bicarbonate solution, dried over sodium sulfate, filtered and concentrated under reduced pressure. The title compound is isolated by triturating the crude product with diisopropyl ether.

Yield: 6.8 g (92% purity, 67% of theory)

HPLC (method 8): Rₜ=5.26 min;

MS (ESI): m/z=535 [M+H]⁺;

¹H-NMR (400 MHz, DMSO-d₆): δ=9.00 (t, 1H), 7.72 (d, 1H), 7.60 (d, 2H), 7.25 (d, 2H), 7.22 (d, 1H), 4.91-4.82 (m, 1H), 4.19 (t, 1H), 3.92-3.81 (m, 5H), 3.67-3.61 (m, 2H), 0.86 (s, 9H), 0.00 (s, 6H).

### Step d): 5-Chloro-N-({(5S)-3-[4-(2-imino-1,3-oxazolidin-3-yl)phenyl]-2-oxo-1,3-oxazolidin-5-yl}methyl)thiophene-2-carboxamide

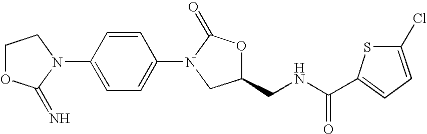

Under argon and at room temperature, 26 ml of hydrogen fluoride solution (10% strength in acetonitrile, 101 mmol, 10 eq.) are added to a solution of 5.4 g (10.1 mmol) of N-[((5S)-3-{4-[(2-{[tert-butyl(dimethyl)silyl]oxy}ethyl)(cyano)amino]phenyl}-2-oxo-1,3-oxazolidin-5-yl)methyl]-5-chlorothiophene-2-carboxamide in 100 ml of THF. The reaction mixture is stirred at room temperature for 2 d and then concentrated under reduced pressure. The residue is dissolved in 150 ml of a dichloromethane/ethanol mixture (15:1), 10 ml of concentrated sodium bicarbonate solution are added and the mixture is stirred at room temperature for 15 min. After phase separation, the organic phase is dried over magnesium sulfate, filtered and concentrated under reduced pressure.

Yield: 4.1 g (96% of theory)

HPLC (method 7): Rₜ=3.73 min;

MS (ESI): m/z=421 [M+H]⁺;

¹H-NMR (400 MHz, DMSO-d₆): δ=8.98 (t, 1H), 7.79 (d, 2H), 7.69 (d, 1H), 7.49 (d, 2H), 7.20 (d, 1H), 6.13 (br. s, 1H), 4.89-4.75 (m, 1H), 4.34 (t, 2H), 4.16 (t, 1H), 3.97 (t, 2H), 3.81 (dd, 1H), 3.60 (t, 2H).

### Example 2

### 5-Chloro-N-({(5S)-3-[4-(2-imino-1,3-oxazolidin-3-yl)phenyl]-2-oxo-1,3-oxazolidin-5-yl}methyl)thiophene-2-carboxamide methanesulfonate

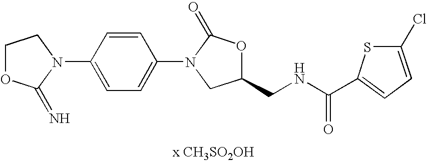

At room temperature, a total of 650 l (10.1 μmol, 2.7 eq.) of methanesulfonic acid are added to a solution of 2.0 g (3.7 mmol) of N-[((5S)-3-{4-[(2-{[tert-butyl(dimethyl)silyl]oxy}ethyl)(cyano)amino]phenyl}-2-oxo-1,3-oxazolidin-5-yl)methyl]-5-chlorothiophene-2-carboxamide (Example 1, step c) in 400 ml of acetonitrile. The reaction mixture is stirred at room temperature for 2 d and then concentrated under reduced pressure, and the title compound is isolated by flash chromatography of the crude product on silica gel (mobile phase: dichloromethane/methanol 20:1→5:1).

Yield: 1.9 g (98% of theory)

HPLC (method 9): Rₜ=3.71 min;

MS (ESI): m/z=421 [M+H]⁺ (free base);

¹H-NMR (400 MHz, DMSO-d₆): δ=9.57 (br. s, 1H), 9.00 (t, 1H), 8.80 (br. s, 1H), 7.71 (d, 2H), 7.69 (d, 1H), 7.54 (d, 2H), 7.20 (d, 1H), 4.93-4.85 (m, 1H), 4.84 (t, 2H), 4.22 (t, 3H), 3.86 (dd, 1H), 3.63 (t, 2H), 2.30 (s, 3H).

The following salts are prepared analogously to the method described in Example 2 by reaction with the appropriate acids:

### Example 3

### 5-Chloro-N-({(5S)-3-[4-(2-imino-1,3-oxazolidin-3-yl)phenyl]-2-oxo-1,3-oxazolidin-5-yl}methyl)thiophene-2-carboxamide trifluoroacetate

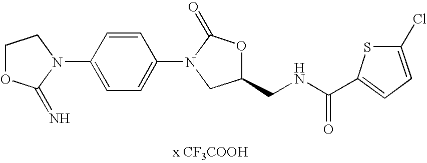

LC-MS (method 3): Rₜ=1.30 min;

MS (ESI): m/z=421 [M+H]⁺ (free base);

¹H-NMR (400 MHz, DMSO-d₆): δ=9.57 (br. s, 1H), 8.99 (t, 1H), 8.79 (br. s, 1H), 7.70 (d, 2H), 7.68 (d, 1H), 7.54 (d, 2H), 7.20 (d, 1H), 4.92-4.84 (m, 1H), 4.84 (t, 2H), 4.21 (t, 3H), 3.86 (dd, 1H), 3.63 (t, 2H).

### Example 4

### 5-Chloro-N-({(5S)-3-[4-(2-imino-1,3-oxazolidin-3-yl)phenyl]-2-oxo-1,3-oxazolidin-5-yl}methyl)thiophene-2-carboxamide hydrochloride

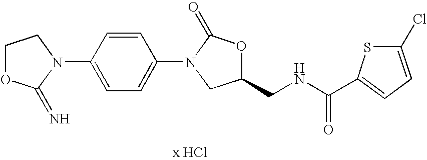

HPLC (method 7): Rₜ=3.73 min;

MS (ESI): m/z=421 [M+H]⁺ (free base);

¹H-NMR (300 MHz, DMSO-d₆): δ=9.59 (br. s, 1H), 9.07 (t, 1H), 8.81 (br. s, 1H), 7.73 (d, 1H), 7.71 (d, 2H), 7.54 (d, 2H), 7.20 (d, 1H), 4.95-4.80 (m, 3H), 4.21 (t, 3H), 3.88 (dd, 1H), 3.62 (t, 2H).

### Example 5

### 5-Chloro-N-({(5S)-3-[4-(2-imino-1,3-oxazinan-3-yl)phenyl]-2-oxo-1,3-oxazolidin-5-yl}methyl)thiophene-2-carboxamide methanesulfonate

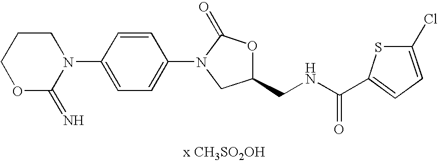

### Step a): N-[(2R)-3-({4-[(3-{[tert-Butyl(dimethyl)silyl]oxy}propyl)amino]phenyl}amino)-2-hydroxypropyl]-5-chlorothiophene-2-carboxamide

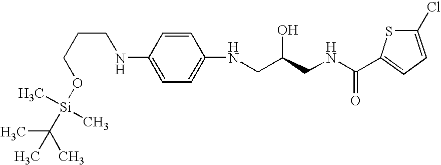

At room temperature and under argon, 95 mg (0.15 mmol, 0.005 eq.) of ytterbium(III) trifluoromethanesulfonate are added to a solution of 8.6 g (30.6 mmol) of the compound from Example 2A in 40 ml of THF. At 60° C., a solution of 4.9 g (22.6 mmol, 0.7 eq.) of the compound from Example 3A in 15 ml of THF is then added dropwise over a period of 2.5 h to the reaction mixture. The mixture is stirred at 60° C. overnight, another 1.7 g (7.9 mmol, 0.3 eq.) of the compound from Example 3A are added and the mixture is stirred for a further 17 h at 60° C. The reaction mixture is then concentrated under reduced pressure, and the crude product is purified by flash chromatography on silica gel (mobile phase: dichloromethane/methanol 100:1→50:1).

Yield: 5.2 g (64% purity, 22% of theory)

LC-MS (method 1): Rₜ=1.88 min;

MS (ESI): m/z=498 [M+H]⁺.

### Step b): N-[((5S)-3-{4-[(3-{[tert-Butyl(dimethyl)silyl]oxy}propyl)amino]phenyl}-2-oxo-1,3-oxazolidin-5-yl)methyl]-5-chlorothiophene-2-carboxamide

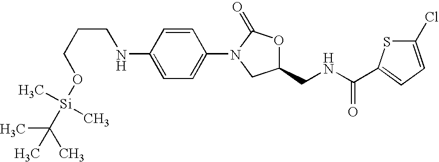

Under argon and at room temperature, 409 mg (3.35 mmol, 0.5 eq.) of 4-N,N-dimethylaminopyridine and 1.6 g (10.1 mmol, 1.5 eq.) of N,N′-carbonyldiimidazole are added to a solution of 5.2 g (64% purity, 6.70 mmol) of N-[(2R)-3-({4-[(3-{[tert-butyl(dimethyl)silyl]oxy}propyl)amino]-phenyl}amino)-2-hydroxypropyl]-5-chlorothiophene-2-carboxamide in 65 ml of THF. The reaction mixture is stirred at room temperature for 1 d, a further 409 mg (3.35 mmol, 0.5 eq.) of 4-N,N-dimethylaminopyridine and 1.6 g (10.1 mmol, 1.5 eq.) of N,N′-carbonyldiimidazole are then added and the mixture is again stirred at room temperature for 1 d. The reaction mixture is then concentrated under reduced pressure, and the title compound is isolated by flash chromatography of the crude product on silica gel (mobile phase: dichloromethane/methanol 300:1→100:1).

Yield: 1.2 g (98% purity, 33% of theory) and also 620 mg (87% purity, 15% of theory)

LC-MS (method 1): Rₜ=2.82 min;

MS (ESI): m/z=524 [M+H]⁺;

¹H-NMR (300 MHz, DMSO-d₆): δ=8.94 (t, 1H), 7.66 (d, 1H), 7.17 (d, 1H), 7.15 (d, 2H), 6.51 (d, 2H), 5.49 (t, 1H), 4.77-5.68 (m, 1H), 4.02 (t, 1H), 3.69 (dd, 1H), 3.65 (t, 2H), 3.53 (t, 2H), 2.99 (qd, 2H), 1.68 (q, 2H), 0.84 (s, 9H), 0.00 (s, 6H).

### Step c): N-[((5S)-3-{4-[(3-{[tert-Butyl(dimethyl)silyl]oxy}propyl)(cyano)amino]phenyl}-2-oxo-1,3-oxazolidin-5-yl)methyl]-5-chlorothiophene-2-carboxamide

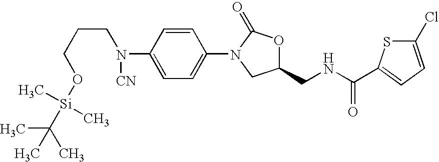

At room temperature, 563 mg (6.70 mmol, 3 eq.) of sodium bicarbonate and 893 μl of cyanogen bromide solution (3 M in dichloromethane, 2.7 mmol, 1.2 eq.) are added to a solution of 1.2 g (2.2 mmol) of N-[((5S)-3-{4-[(3-{[tert-butyl(dimethyl)silyl]oxy}propyl)amino]phenyl}-2-oxo-1,3-oxazolidin-5-yl)methyl]-5-chlorothiophene-2-carboxamide in 7.5 ml of THF. The reaction mixture is stirred at 40° C. for 1 d, and water and dichloromethane are then added. After phase separation, the organic phase is washed with saturated aqueous sodium bicarbonate solution, dried over sodium sulfate, filtered and concentrated under reduced pressure. The title compound is isolated by recrystallization of the crude product from ethyl acetate.

Yield: 814 mg (66% of theory)

LC-MS (method 6): Rₜ=2.73 min;

MS (ESI): m/z=549 [M+H]⁺;

¹H-NMR (300 MHz, DMSO-d₆): δ=8.96 (t, 1H), 7.67 (d, 1H), 7.56 (d, 2H), 7.19 (d, 1H), 7.17 (d, 2H), 4.87-4.76 (m, 1H), 4.15 (t, 1H), 3.81 (dd, 1H), 3.75-3.64 (m, 4H), 3.59 (t, 2H), 1.84 (q, 2H), 0.85 (s, 9H), 0.02 (s, 6H).

### Step d): 5-Chloro-N-({(5S)-3-[4-(2-imino-1,3-oxazinan-3-yl)phenyl]-2-oxo-1,3-oxazolidin-5-yl}methyl)thiophene-2-carboxamide methanesulfonate

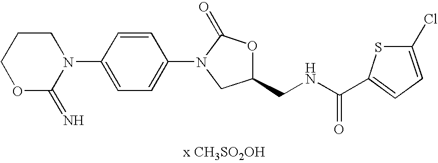

At room temperature, 201 μl (3.09 mmol, 2.1 eq.) of methanesulfonic acid are added to a suspension of 808 mg (1.47 mmol) of N-[((5S)-3-{4-[(3-{[tert-butyl(dimethyl)silyl]oxy}propyl)(cyano)amino]phenyl}-2-oxo-1,3-oxazolidin-5-yl)methyl]-5-chlorothiophene-2-carboxamide in 159 ml of acetonitrile. The reaction mixture is stirred at room temperature for 2 d and then concentrated under reduced pressure, and the title compound is isolated by flash chromatography of the crude product on silica gel (mobile phase: dichloromethane/methanol 5:1→4:1).

Yield: 400 mg (51% of theory)

HPLC (method 7): Rₜ=3.75 min;

MS (ESI): m/z=435 [M+H]⁺ (free base);

¹H-NMR (400 MHz, DMSO-d₆): δ=9.00 (t, 1H), 8.74 (br. s, 1H), 7.73 (d, 2H), 7.70 (d, 1H and also br. s, 1H), 7.55 (d, 2H), 7.20 (d, 1H), 4.94-4.85 (m, 1H), 4.60 (t, 2H), 4.22 (t, 1H), 3.86 (dd, 1H), 3.62 (t, 4H), 2.30 (s, 3H), 2.27 (q, 2H).

### Example 6

### 5-Chloro-N-[((5S)-3-{4-[2-(cyanoimino)-1,3-oxazolidin-3-yl]phenyl}-2-oxo-1,3-oxazolidin-5-yl)methyl]thiophene-2-carboxamide

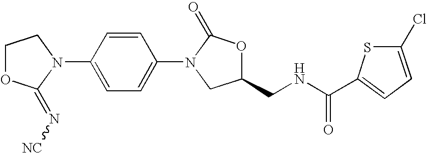

The title compound is obtained as a byproduct in the reaction of 5-chloro-N-[((5S)-3-{4-[(2-hydroxyethyl)amino]phenyl}-2-oxo-1,3-oxazolidin-5-yl)methyl]thiophene-2-carboxamide (Example 7, step a) with excess cyanogen bromide solution (3 M in dichloromethane, 50 eq.) in dichloromethane and subsequent basic work-up with saturated aqueous sodium bicarbonate solution (cf. Example 1, step c).

HPLC (method 7): Rₜ=3.95 min;

MS (ESI): m/z=446 [M+H]⁺;

¹H-NMR (400 MHz, DMSO-d₆): δ=8.97 (t, 1H), 7.68 (d, 1H), 7.63-7.53 (m, 4H), 7.19 (d, 1H), 4.89-4.80 (m, 1H), 4.73 (t, 2H), 4.25 (t, 2H), 4.18 (t, 1H), 3.84 (dd, 1H), 3.61 (t, 2H).

### Example 7

### N-[((5S)-3-{4-[2-(Benzoylimino)-1,3-oxazolidin-3-yl]phenyl}-2-oxo-1,3-oxazolidin-5-yl)methyl]-5-chlorothiophene-2-carboxamide

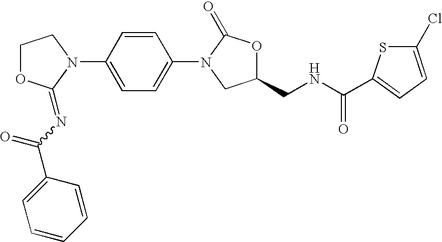

### Step a): 5-Chloro-N-[((5S)-3-{4-[(2-hydroxyethyl)amino]phenyl}-2-oxo-1,3-oxazolidin-5-yl)methyl]thiophene-2-carboxamide

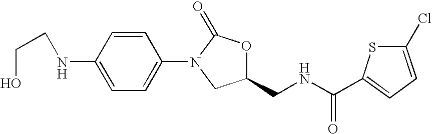

At 0° C., 3.5 ml of tetra-n-butylammonium fluoride solution (1 M in THF, 3.53 mmol, 2 eq.) are added to a solution of 900 mg (1.76 mmol) of N-[((5S)-3-{4-[(2-{[tert-butyl(dimethyl)silyl]oxy}-ethyl)amino]phenyl}-2-oxo-1,3-oxazolidin-5-yl)methyl]-5-chlorothiophene-2-carboxamide in 25 ml of THF. The reaction mixture is stirred at room temperature for 2 h and then concentrated under reduced pressure. The title compound is isolated by flash chromatography of the crude product on silica gel (mobile phase: dichloromethane/ethanol 20:1).

Yield: 365 mg (52% of theory)

LC-MS (method 3): Rₜ=1.62 min;

MS (ESI): m/z=396 [M+H]⁺;

¹H-NMR (400 MHz, DMSO-d₆): δ=8.95 (t, 1H), 7.69 (d, 1H), 7.20 (d, 2H), 7.18 (d, 1H), 6.58 (d, 2H), 5.45 (t, 1H), 4.79-4.71 (m, 1H), 4.66 (t, 1H), 4.06 (t, 1H), 3.72 (dd, 1H), 3.57 (t, 2H), 3.53 (qd, 2H), 3.07 (qd, 2H).

### Step b): N-[((5S)-3-{4-[[(Benzoylamino)carbonothioyl](2-hydroxyethyl)amino]phenyl}-2-oxo-1,3-oxazolidin-5-yl)methyl]-5-chlorothiophene-2-carboxamide

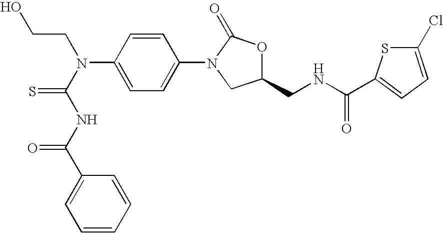

Under argon and at room temperature, 200 μl (1.52 mmol, 1.2 eq.) of benzoyl isothiocyanate are added to a solution of 500 mg (1.26 mmol) of 5-chloro-N-[((5S)-3-{4-[(2-hydroxyethyl)amino]-phenyl}-2-oxo-1,3-oxazolidin-5-yl)methyl]thiophene-2-carboxamide in 20 ml of acetone. The reaction mixture is stirred at room temperature for 3 d and then concentrated under reduced pressure.

The title compound is isolated by trituration of the residue with diisopropyl ether.

Yield: 730 mg (86% purity, 89% of theory)

HPLC (method 7): Rₜ=4.14 min;

MS (ESI): m/z=559 [M+H]⁺.

### Step c): N-[((5S)-3-{4-[2-(Benzoylimino)-1,3-oxazolidin-3-yl]phenyl}-2-oxo-1,3-oxazolidin-5-yl)methyl]-5-chlorothiophene-2-carboxamide

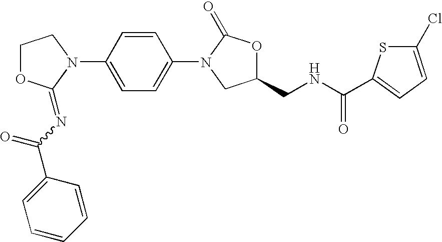

Under argon and at room temperature, 419 mg (1.64 mmol, 1.5 eq.) of 2-chloro-1-methylpyridinium iodide are added to a suspension of 720 mg (1.09 mmol) of N-[((5S)-3-{4-[[(benzoylamino)carbonothioyl](2-hydroxyethyl)amino]phenyl}-2-oxo-1,3-oxazolidin-5-yl)methyl]-5-chlorothiophene-2-carboxamide and 390 μl (2.73 mmol, 2.5 eq.) of triethylamine in 12 ml of acetonitrile. The reaction mixture is stirred at room temperature for 17 h, and water and dichloromethane are then added. After phase separation, the aqueous phase is re-extracted with dichloromethane. The combined organic phases are washed with water, dried over sodium sulfate, filtered and concentrated under reduced pressure. The title compound is isolated by preparative RP-HPLC.

Yield: 213 mg (39% of theory) and also 162 mg (90% purity, 26% of theory)

LC-MS (method 2): Rₜ=2.34 min;

MS (ESI): m/z=525 [M+H]⁺;

¹H-NMR (400 MHz, DMSO-d₆): δ=8.97 (t, 1H), 7.96 (d, 2H), 7.77 (d, 2H), 7.69 (d, 1H), 7.61 (d, 2H), 7.54 (t, 1H), 7.45 (t, 2H), 7.19 (d, 1H), 4.89-4.81 (m, 1H), 4.62 (t, 2H), 4.24-4.15 (m, 3H), 3.86 (dd, 1H), 3.61 (t, 2H).

### B. EVALUATION OF THE PHARMACOLOGICAL ACTIVITY

The compounds according to the invention act in particular as selective inhibitors of blood coagulation factor Xa and do not, or only at significantly higher concentrations, inhibit other serine proteases, such as plasmin or tyypsin.

Inhibitors of blood coagulation factor Xa are referred to as being “selective” if the IC₅₀ values for factor Xa inhibition are smaller by a factor of at least 100 compared with the IC₅₀ values for the inhibition of other serine proteases, in particular plasmin and trypsin, where, with a view to the test methods for selectivity, reference is made to the test methods described below of Examples B.a.1) and B.a.2).

The advantageous pharmacological properties of the compounds according to the invention can be determined by the following methods:

### a) Test Descriptions (In Vitro)

### a.1) Determination of the Factor Xa Inhibition:

The enzymatic inhibition of human factor Xa (FXa) is measured using the conversion of a chromogenic substrate specific for FXa. Factor Xa cleaves p-nitroaniline from the chromogenic substrate. The determinations are carried out in microtiter plates as follows:

The test substances, in various concentrations, are dissolved in DMSO and incubated for 10 minutes at 25° C. with human FXa (0.5 nmol/l dissolved in 50 mmol/l of Tris buffer [C,C,C-tris(hydroxymethyl)aminomethane], 150 mmol/l of NaCl, 0.1% BSA [bovine serum albumin], pH 20=8.3). Pure DMSO is used as control. The chromogenic substrate (150 μmol/l Pefachrome® FXa from Pentapharm) is then added. After an incubation time of 20 minutes at 25° C., the extinction at 405 nm is determined. The extinctions of the test mixtures containing test substance are compared with the control mixtures without test substance, and the IC₅₀ values are calculated from these data.

Representative activity data from this test are listed in Table 1 below:

TABLE 1

| Example No. | IC₅₀ [nM] |
|---|---|
| 1 | 1.1 |
| 5 | 3.1 |
| 6 | 16 |

### a.2) Determination of the Selectivity:

To assess selective FXa inhibition, the test substances are examined for their inhibition of other human serine proteases such as trypsin and plasmin. To determine the enzymatic activity of trypsin (500 mU/ml) and plasmin (3.2 nmol/l), these enzymes are dissolved in Tris buffer (100 mmol/l, 20 mmol/l CaCl₂, pH=8.0) and incubated with test substance or solvent for 10 minutes. The enzymatic reaction is then started by adding the corresponding specific chromogenic substrates (Chromozym Trypsin® and Chromozym Plasmin®; from Roche Diagnostics) and the extinction at 405 nm is determined after 20 minutes. All determinations are carried out at 37° C. The extinctions of the test mixtures containing test substance are compared with the control samples without test substance, and the IC₅₀ values are calculated from these data.

### a.3) Determination of the Anticoagulant Action:

The anticoagulant action of the test substances is determined in vitro in human and rabbit plasma. To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 2500 g for 10 minutes. The supernatant is pipetted off. The prothrombin time (PT, synonyms: thromboplastin time, quick test) is determined in the presence of varying concentrations of test substance or the corresponding solvent using a commercial test kit (Hemoliance® RecombiPlastin, from Instrumentation Laboratory). The test compounds are incubated with the plasma at 37° C. for 3 minutes. Coagulation is then started by addition of thromboplastin, and the time when coagulation occurs is determined. Concentration of test substance which effects a doubling of the prothrombin time is determined.

### b) Determination-of the Antithrombotic Activity (In Vivo)

### b.1) Arteriovenous Shunt Model (Rabbit):

Fasting rabbits (strain: Esd: NZW) are anaesthetized by intramuscular administration of a Romsun/

Ketavet solution (5 mg/kg and 40 mg/kg, respectively). Thrombus formation is initiated in an arteriovenous shunt in accordance with the method described by C. N. Berry et al. [Semin. Thromb. Hemost. 1996, 22, 233-241]. To this end, the left jugular vein and the right carotid artery are exposed. The two vessels are connected by an extracorporeal shunt using a vein catheter of a length of 10 cm. In the middle, this catheter is attached to a further polyethylene tube (PE 160, Becton Dickenson) of a length of 4 cm which contains a roughened nylon thread which has been arranged to form a loop, to form a thrombogenic surface. The extracorporeal circulation is maintained for 15 minutes. The shunt is then removed and the nylon thread with the thrombus is weighed immediately. The weight of the nylon thread on its own was determined before the experiment was started. Before extracorporeal circulation is set up, the test substances are administered either intravenously via an ear vein or orally using a pharyngeal tube.

### C. WORKING EXAMPLES OF PHARMACEUTICAL COMPOSITIONS

The compounds according to the invention can be converted into pharmaceutical preparations in the following ways:

### Tablet:

### Composition:

100 mg of the compound according to the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

### Production:

The mixture of the compound according to the invention, lactose and starch is granulated with a 5% strength solution (m/m) of the PVP in water. The granules are dried and then mixed with the magnesium stearate for 5 minutes. This mixture is compressed using a conventional tablet press (see above for the dimensions of the tablet). A compressive force of 15 kN is used as a guideline for the compression.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

### Production:

The Rhodigel is suspended in ethanol, and the compound according to the invention is added to the suspension. The water is added while stirring. The mixture is stirred for about 6 h until the swelling of the Rhodigel is complete.

Solution which can be Administered Orally:

### Composition:

500 mg of the compound according to the invention, 2.5 g of polysorbate and 97 g of polyethylene glycol 400.20 g of oral solution correspond to a single dose of 100 mg of the compound according to the invention.

### Production:

The compound according to the invention is suspended in the mixture of polyethylene glycol and polysorbate with stirring. Stirring is continued until the compound according to the invention has dissolved completely.

i.v. Solution:

The compound according to the invention is, at a concentration below saturation solubility, dissolved in a physiologically acceptable solvent (for example isotonic saline, glucose solution 5% and/or PEG 400 solution 30%). The solution is subjected to sterile filtration and filled into sterile and pyrogen-free injection containers.

1. A compound of the formula (I) 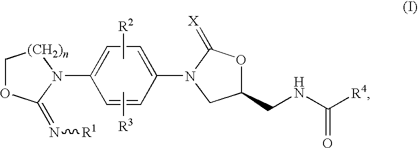
in which
R¹ represents hydrogen, hydroxyl, cyano, (C₁-C₆)-alkyl, (C₁-C₆)-alkanoyl, benzoyl or heteroaroyl,
R² and R³ are identical or different and independently of one another represent hydrogen, fluorine, chlorine, cyano, (C₁-C₄)-alkyl, cyclopropyl, trifluoromethyl, hydroxyl, (C₁-C₄)-alkoxy, trifluoromethoxy, amino, mono- or di-(C₁-C₄)-alkylamino,
R⁴ represents phenyl, pyridyl, pyrimidinyl, pyrazinyl, thienyl, furyl or pyrrolyl which may in each case be mono- or disubstituted by identical or different substituents selected from the group consisting of halogen, cyano, (C₁-C₄)-alkyl, which for its part may be substituted by hydroxyl or amino, (C₁-C₄)-alkoxy, ethynyl, cyclopropyl and amino,
n represents the number 1, 2 or 3,
and
x represents O or N—R⁵, where
R⁵ represents hydrogen, cyano, (C₁-C₆)-alkyl or phenyl, where phenyl may be mono- or disubstituted by identical or different substituents selected from the group consisting of halogen, cyano, trifluoromethyl, (C₁-C₄)alkyl and (C₁-C₄)-alkoxy,
and salts, solvates and solvates of the salts thereof.

2. The compound of the formula (I) as claimed in claim 1 in which
R¹ represents hydrogen, hydroxyl, cyano, (C₁-C₆)-alkyl, (C₁-C₆)-alkanoyl, benzoyl or heteroaroyl,
R² and R³ are identical or different and independently of one another represent hydrogen, fluorine, chlorine, cyano, (C₁-C₄)-alkyl, cyclopropyl, trifluoromethyl, hydroxyl, (C₁-C₄)-alkoxy, trifluoromethoxy, amino, mono- or di-(C₁-C₄)-alkylamino,
R⁴ represents phenyl, pyridyl, pyrimidinyl, pyrazinyl, thienyl, furyl or pyrrolyl which may in each case be mono- or disubstituted by identical or different substituents selected from the group consisting of halogen, cyano, (C₁-C₄)-alkyl, which for its part may be substituted by hydroxyl or amino, (C₁-C₄)-alkoxy, ethynyl, cyclopropyl and amino,
n represents the number 1, 2 or 3,
and
x represents O or N—R⁵, where
R⁵ represents hydrogen, cyano or (C₁-C₆)-alkyl,
and salts, solvates and solvates of the salts thereof.

3. The compound of the formula (I) as claimed in claim 1 in which
R¹ represents hydrogen, hydroxyl, cyano or methyl,
R² represents hydrogen,
R³ represents hydrogen, fluorine, chlorine, cyano, methyl or methoxy,
R⁴ represents a group of the formula 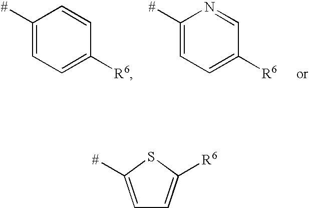
in which
R⁶ represents fluorine, chlorine, methyl or ethynyl
and
# denotes the point of attachment to the carbonyl group,
n represents the number 1 or 2,
and
x represents O,
and salts, solvates and solvates of the salts thereof.

4. The compound of the formula (I) as claimed in claim 1 in which
R¹ represents hydrogen,
R² represents hydrogen,
R³ represents hydrogen, fluorine or methyl,
R⁴ represents a group of the formula 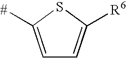
in which
R⁶ represents fluorine, chlorine or methyl
and
# denotes the point of attachment to the carbonyl group,
n represents the number 1 or 2,
and
x represents O,
and salts, solvates and solvates of the salts thereof.

5. A process for preparing compounds of the formula (I), as defined in claim 1, in which R¹ represents hydrogen and X represents oxygen, characterized in that compounds of the formula (II) 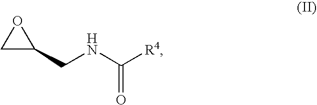
in which R⁴ has the meaning given in claim 1,
are reacted in an inert solvent with a compound of the formula (III) 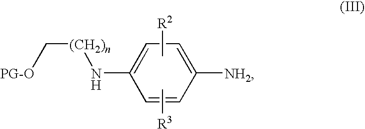
in which n, R² and R³ have the meanings given in claim 1
and
PG represents a hydroxyl protective group,
to give compounds of the formula (IV) 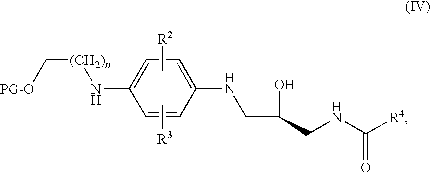
in which n, PG, R², R³ and R⁴ have the meanings given above,
these are then reacted in an inert solvent with a carbonic acid equivalent to give compounds of the formula (V) 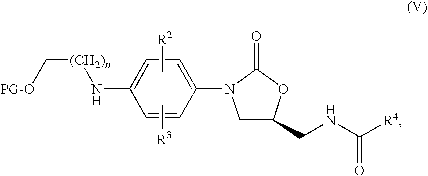
in which n, PG, R², R³ and R⁴ have the meanings given above, and then either
[A] by removal of the protective group PG converted into compounds of the formula (VI) 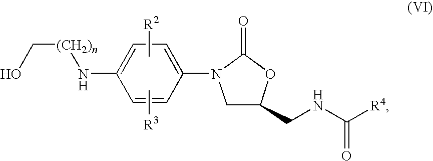
in which n, R², R³ and R⁴ have the meanings given above,
and the compounds of the formula (VI) are then in an inert solvent in the presence of an acid converted with cyanogen bromide into compounds of the formula (I-A) 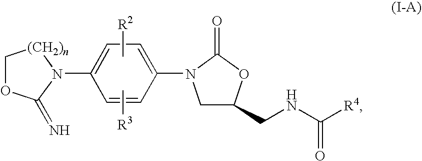
in which n, R², R³ and R⁴ have the meanings given above, or
[B] initially reacted in an inert solvent with cyanogen bromide to give compounds of the formula (VII) 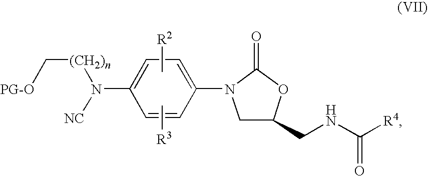
in which n, PG, R², R³ and R⁴ have the meanings given above,
then by removal of the protective group PG converted into compounds of the formula (VIII) 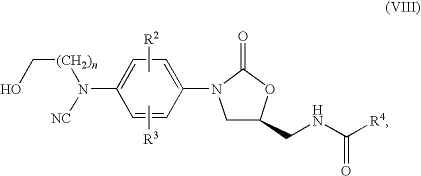
in which n, R², R³ and R⁴ have the meanings given above, and the compounds of the formula (VIII) are then cyclized in an inert solvent in the presence of an acid to give compounds of the formula (I-A)
and the compounds of the formula (I-A) are, if appropriate, converted with the appropriate (i) solvents and/or (ii) bases or acids into their solvates, salts and/or solvates of the salts.

6. A process for preparing compounds of the formula (I), as defined in claim 1, in which R¹ represents hydrogen and X represents NH, characterized in that, starting with compounds of the formula (IV) 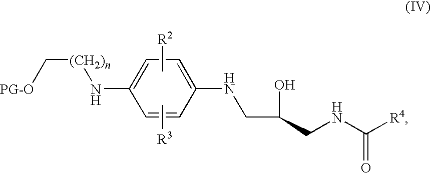
in which n, R², R³ and R⁴ have the meanings given in claim 1
and
PG represents a hydroxyl protective group,
initially by introducing a second hydroxyl protective group PG, compounds of the formula (IX) 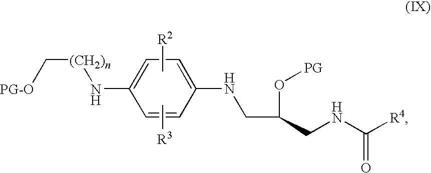
in which n, PG, R², R³ and R⁴ have the meanings given above, are prepared, which are then, in an inert solvent using cyanogen bromide, converted into compounds of the formula (X) 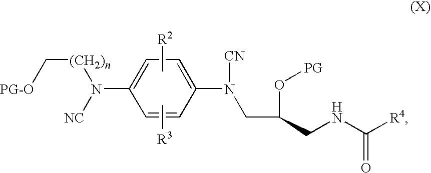
in which n, PG, R², R³ and R⁴ have the meanings given above,
then, by removing the protective groups PG, converted into compounds of the formula (XI) 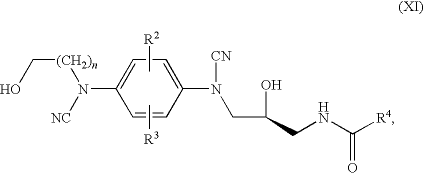
in which n, R², R³ and R⁴ have the meanings given above,
and these are cyclized in an inert solvent in the presence of an acid to compounds of the formula (I-B) 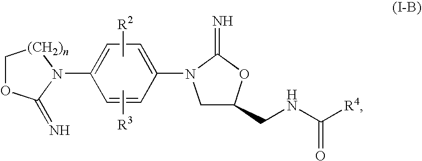
in which n, R², R³ and R⁴ have the meanings given above,
and the compounds of the formula (I-B) are, if appropriate, converted with the appropriate (i) solvents and/or (ii) bases or acids into their solvates, salts and/or solvates of the salts.

7. A compound of the formula (I) as defined in claim 1 for the treatment and/or prophylaxis of diseases.

8. (canceled)

9. The use of a compound of the formula (I) as defined in claim 1 for preventing blood coagulation in vitro.

10. A pharmaceutical composition, comprising a compound of the formula (I) as defined in claim 1 in combination with an inert nontoxic, pharmaceutically acceptable auxiliary.

11. The pharmaceutical composition of claim 10, further comprising another active compound.

12. The pharmaceutical composition as claimed in claim 10 for the treatment and/or prophylaxis of thromboembolic disorders.

13. A method for the treatment and/or prophylaxis of thromboembolic disorders in humans and animals, comprising administering an anticoagulatorilly effective amount of at least one compound of the formula (I) as defined in claim 1.

14. A method for preventing blood coagulation in vitro, comprising contacting a blood sample with an anticoagulatorilly effective amount of a compound of the formula (I) as defined in claim 1.

15. A method for the treatment and/or prophylaxis of thromboembolic disorders in humans and animals, comprising administering an anticoagulatorilly effective amount of a pharmaceutical composition of claim 10.
